(19) United States
(12) Patent Application Publication
(10) Pub. No.: US 2006/0280809 A1
(43) Pub. Date: Dec. 14, 2006

(54) Anti-infective iodine based compositions for otic and nasal use

(76) Inventors: Adele K. Leshchiner, Cresskill, NJ (US); Nancy E. Larsen, Highland Mills, NY (US); Edward G. Parent, North Bergen, NJ (US)

Correspondence Address: Sheldon Palmer;c/o Galvin & Palmer, Suite 1400, 630 Third Avenue, New York, NY, 10017, US

(21) Appl. No.: 11/452,372

(22) Filed: Jun. 14, 2006

Related U.S. Application Data
(60) Provisional application No. 60/689,946, filed on Jun. 14, 2005.

(51) Int. Cl. A61K 33/36; A61K 31/727; A61K 31/16; A61K 31/728; A61K 31/737; A61K 31/573; A61K 36/899; A61K 36/47; A61K 36/28; A61K 36/72; A61K 36/185

(52) U.S. Cl. 424/672; 424/731; 424/750; 424/764; 424/765; 424/778; 514/54; 514/56; 514/171; 514/629

(57) ABSTRACT

Otic and nasal compositions containing any iodine-containing derivatives, including, free iodine and iodoform, are disclosed. lodoform is a potent germicidal agent which provides anti-infective benefits. The composition also contains one or more anti-inflammatory agents and one or more natural or synthetic compounds which provide analgesic benefits. The composition preferably also contains one or more natural or synthetic compounds which provides aromatic benefits. The composition may be utilized to treat otic and nasal conditions, including otitis media, by topically applying the composition to the affected tissue.

### CROSS-REFERENCE TO RELATED APPLICATIONS

This is a non-provisional application based on and claiming the benefit of the filing date of provisional application Ser. No. 60/689,946; filed Jun. 14, 2005.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### REFERENCE TO A MICROFICHE APPENDIX

Not Applicable

### BACKGROUND OF THE INVENTION

1. Field of the Invention

The invention relates to the use of iodine and iodine compounds and compositions containing such iodine and iodine compounds for the treatment of otitis media and paranasal sinusitis.

2. Description of the Related Art

The growing public health problem of disease-causing microbes that are resistant to drug therapy is due largely to the increasing use of antibiotics. According to the U.S. FDA, childhood ear infection is one of the many diseases that have become hard to treat with antibiotic drugs because of antibiotic resistant bacteria and antibiotic-resistant microorganisms. Most cases of otitis media (OM) are caused by one of several major pathogens, Streptococcus pneumonia, Haemophilus influenza, Moraxella catarrhalia, Staphylococcus aureus, Staphylococcus epidermidis, or Pseudomonas aeruginosa. There is thus an urgent need to develop new, non-antibiotic approaches to prevent and manage these diseases.

The use of oral antibiotics to treat otic and nasal infection in children represents the current state of the art in this field. The use of oral antibiotics to treat otic infection in children has limited efficacy and creates a serious risk of pathogen resistance to the orally administered antibiotics.

Otic and nasal infections are often accompanied by inflammation and pain in the infected otic and nasal tissues. There is a need for otic and nasal pharmaceutical compositions that combine the broad anti-infective activity of iodine-based compounds with the anti-inflammatory and analgesic activity of natural and synthetic oils and extracts as well as steroidal and non-steroidal anti-inflammatory agents.

Another therapy for treatment of moderate otitis extema is the application of antibiotic drops to the ear or oral antibiotic therapy. Administration of antibiotics is associated with various disadvantages including the risk of ototoxicity, the risk of overuse of antibiotics and the growth of drug-resistant bacteria.

It is known in the art to use iodine and iodine derivatives to treat oral, dermal, and other infections. Iodine and iodine derivatives possess potent antimicrobial activity and the local delivery of these agents to the site of infection is known to effectively treat, eliminate, and/or prevent the growth of microorganisms. The use of iodoform and iodine-based agents to manage infections is also well known in the dental and wound care areas. For example, a dental paste containing iodoform is marketed by Neo Dental International under the name Vitapex. Iodoform is also been used in wound treatment products including wound packing products containing 5% iodoform.

Hei et al., U.S. Pat. No. 6,663,902 describes the use of iodine/iodine containing substances to clean, sanitize, deodorize, and disinfect animate and inanimate surfaces, and suggests use in the veterinary field to treat ear and eye disease, but there is no suggestion to use them in treating otitis media or otitis extema.

Dixon, et al., U.S. Pat. No. 5,554,361 discloses the use of processed iodine-solutions for slin and hair treatment and compositions used to relieve pain and infection associated with the ear and auditory canals. Dixon also describes the use of processed iodine-solutions to treat ingrown toenail pain, blood circulation and removal of wrinkles.

Other, non-iodine based products for use in this field are disclosed in Cagle, et al., U.S. Pat. No. 6,716,830 which describes ophthalmic antibiotic compositions containing moxifloxacin. There are a number of other products approved for human and veterinary use—an antibiotic ear drop for treatment of ear infection/Otitis Media, one such product is known as MOMETAMX Otic Suspension for Dogs; there is a product for human use; Cipro-HC (ciprofloxacin and hydrocortisone).

BAYTRIL®OTIC is an ear drop products (ototopical use) containing enrofloxacin/silver sulfadiazine emulsion for the treatment of Otitis Extema in the veterinary field. Lim et al., U.S. Pat. No. 6,716,813 describes the use of non-iodine based, non-antibiotic anti-microbial proteins and peptides in an ear drop for the treatment of otitis media.

Spencer, H. N. Iodoform and Alum in Aural Therapeutics. American Journal of Otology, 1879; 1; 287-290 describes the use of iodoform in treating certain “papillary growths” in the ear.

Other references of interest are:

Lawrence, J. C., The use of iodine as an antiseptic agent. J. Wound Care 7(8):421-5 (1998).

Burks, R. I., Povidone-iodine solutions in wound treatment. Phys. Ther 78(2):212-8 (1998).

Nakagawa T. Et al. The efficacy of povidone-iodine products against periodontopathic bacteria. Dermatology 212 Suppl 1: 109-11 (2006).

Gottardi, W. Iodine and iodine compounds In S.S. Block (ed) Disinfection, Sterilization and Preservation, 4^{th} ed.(1991) pp 152-166. Lea & Febiger, Philadelphia, Pa.

Kruse, W. C. Halogen action on bacteria, viruses and protozoa, p 113-137. Proceedings of the National Special Conference on Disinfection. ASCE, Amherst, Mass. (1970).

Chang, S. L. Modem concept of disinfection. J. Sanit. Eng. Div. Proc. ASCE 97:689 (1971).

Apostolov, K. The effects of iodine on the biological activities of myxoviruses. J. Hyg. 84:381-388 (1980).

Tang, L. J., et al. Bacteriostasis of iodoform in vivo and in vitro. Di Yi Jun Yi Da Xue Xue Bao Nov. 23, 2003 (11):1207-10.

Thomas, A. M., et al. Elimination of infection in pulpectomized teeth: A short-term study using lodoform paste. J. Endod. 20(5):233-5 (1994).

Nurko, C. Et al. Evaluation of a calcium hydroxide/lodoform paste (Vitapex) in root canal therapy for primary teeth. J. Clin. Pediatric Dent 23(4):289-294 (1999).

### BRIEF SUMMARY OF THE INVENTION

The present invention is directed to the provision of topical antimicrobial, non-antibiotic, pharmaceutical compositions for the treatment of otic and nasal infections, particularly otitis media, and to methods of treating otic and nasal infections by applying those compositions to the affected tissues. The compositions are based on the use of polyvinlypyrolidone-iodine (PVP-I).

The compositions of the present invention may also contain one or more anti-inflammatory, analgesic and antiseptic agents. The compositions of the invention may also contain substances for masking the odor of iodine.

The mode of action of iodine in treating the noted conditions differs from that of antibiotics in that it is a general microbiocidal substance resulting from the oxidative interaction with vital enzymes within the cell membrane or cell protoplasm.

Many iodine-containing and iodine-releasing substances including iodoform and iodine tinctures are poorly suited for use in the ear or nasal-sinus cavities due to their strong, irritating odor. It is difficult to adequately mask the odor, and therefore many iodine preparations, including iodoform and solutions of iodine-iodide are not tolerated for use in the ear or nasal-sinus cavity in the pediatric and veterinary market despite the use of odor-masking agents. The iodophor polyvinylpyrolidone-iodine (PVP-I), known commercially as povidone-iodine (10% PVP-I in an aqueous buffered salt solution in which the total available iodine is 1%,) and Povadyne® (the straight form of polyvinlypyrolidone containing 10% iodine in powder form) have greatly reduced odor, and improved tolerability when applied topically to dermal and other tissue surfaces. The invention provides a composition containing PVP-I in a vehicle of hyaluronic acid, preferably having a molecular weight of about 500,000 to 6,000,000 and containing additionally natural oils, natural extracts, and solvents which provide a suitable product for use in the ear and nasal-sinus cavities with improved odor and enhanced physical properties such as viscosity.

The invention provides, in one embodiment thereof, topical pharmaceutical compositions for use in treating and relieving the symptoms of ear and sinus infections, i.e., otitis and sinusitis. The compositions all comprise, as the therapeutic component, PVP-I in an amount effective to reduce the growth of infection causing microbes and a pharmaceutically acceptable carrier therefor.

In one embodiment thereof, the invention provides a pharmaceutical preparation for the treatment of otitis and sinusitis, comprising PVP-I in an amount effective to reduce the growth of microbes, and a pharmaceutically acceptable liquid carrier therefor.

In another embodiment, the invention provides a method for the treatment of ear and sinus infection comprising administering to a mammal afflicted with an ear or sinus infection, a therapeutically effective amount of a composition according to the invention.

In yet other embodiments, the invention provides a method for the treatment of ear and sinus infections comprising administering to a mammal afflicted with an ear or sinus infection, a therapeutically effective amount of a composition comprising PVP-Iodine and a pharmaceutically effective carrier therefor, wherein, when the infection is a sinus infection, the composition is administered intranasally, and when the infection is an ear infection, the composition is administered by application of liquid droplets of the composition directly into the ear canal.

The composition according to the invention typically comprises, by weight, 5-50% PVP-I, preferably, 10-20%, more preferably, 10-15%, and in a preferred embodiment, 12%; 0.5-10% of an excipient, preferably 2-5%, more preferably, 1-3%, and in a preferred embodiment, 1%; and 50-90% of a vehicle, preferably, 50-80%, more preferably, 60-75%, and in a preferred embodiment, 77%.

### BRIEF DESCRIPTION OF THE SEVERAL VIEWS OF THE DRAWINGS

Not Applicable

### DETAILED DESCRIPTION OF THE INVENTION

The invention is described in more detail by reference to the following examples, where all parts given are by weight, unless otherwise indicated.

### EXAMPLES

### Example 1

In this example there are prepared 100 grams of a formulation for ear and nose drops by mixing 97 gm of povidone-iodine solution (povidone-iodine is a solution consisting of 10% polyvinylpyrolidone-iodine complex and 90% aqueous salt solution and containing 1% free iodine) with 1 gm of an 0.1%, by weight, hyaluronic acid (of bacterial origin) solution in physiological saline, and 2 gm of calendula extract as excipients to produce a uniform solution for application to that portion of the animal to be treated therewith.

### Example 2

This example illustrates the preparation of 100 grams of the formulation of example 1, using 99 gm of the povidone-iodine solution and 1 gm of eugenol as the excipient.

### Example 3

This example illustrates the preparation of 100 grams of the formulation of example 1, using 99 gm of the povidone-iodine solution with 1 gm of clove oil as the excipient.

### Example 4

This example illustrates the preparation of 100 gm of the formulation of example 1, using 99 gm of the povidone-iodine solution with 1 gm of sea-buckhorn oil as the excipient.

### Example 5

This example illustrates the preparation of 100 gm of the formulation of example 1, using 99 gm of the povidone-iodine solution with 1 gm of herbal tincture of propolis as the excipient.

### Example 6

This example demonstrates the use of the povidone-iodine preparation described in example 1 to treat a chronic canine ear infection diagnosed by a veterinarian. The ears of the dog to be treated showed symptoms of infection including pain, exudate, odor, colored discharge, head shaking and had previously been unsuccessfully treated several times with an antibiotic-steroid ointment together with intense cleansing of the ears and ear canals. 1-2 drops of the povidone-iodine preparation were instilled into the ear using a long tipped plastic pipette, and the ears were gently massaged for several seconds. This treatment was repeated 2 days later. Symptoms of infection were reduced within several days and were completely resolved within 7-10 days (absence of pain, absence of odor, absence of discharge and exudates). The dog was evaluated by the veterinarian several months later and found to be free of the symptoms and of infection/inflammation.

### Example 7

A 5 year old human female had been treated repeatedly with oral antibiotic therapy in an attempt to alleviate and cure the ear infections (otitis media) which had she had been experiencing over the previous 3.5 years (since she was 1.5 years old). The 5 year old girl was treated with a composition containing comprised of 8% polyvinylpyrolidone-iodine complex (PVP-I) containing 0.8% total iodine in the final composition—3% glycerin and saline solution composed of 0.15M sodium chloride dissolved in water and 2% of a 20% solution of rose oil in polysorbate (Tween 80). The composition was prepared by mixing 8 ml of PVP-I (complex containing 10% iodine complexed with polyvinylpyrolidone) with 3 ml of glycerin and 87 ml of 0.15M sodium chloride solution and 2 ml of a 20% solution of rose oil in polysorbate (Tween 80). Two drops were applied to each affected ear. Relief from auricular pain occurred within 24 hours after instillation of the iodine-containing ear drop treatment. Several days after treatment a physician examined the girl and did not observe signs of infections. Several months later, the infection recurred, and the child was treated with the same iodine composition, this time the ear drops were applied three times over a period of 5 days (drops were applied on day 1, day 3 and day 5). The child recovered from the ear infection and remained free of auricular infection (otitis media and/or otitis externa) for several years.

### Example 8

A 2 year old human female (sister of the 5 year old described in Example 10) afflicted with otitis media was treated with a composition containing The composition was prepared by mixing 9.5 ml of PVP-I (complex containing 10% iodine complexed with polyvinylpyrolidone) with 3 ml of glycerin and 85.5 ml of 0.15M sodium chloride solution and 2 ml of a 20% solution of rose oil in polysorbate (Tween 80). The treatment was applied as follows: 2 drops per affected ear on the first day, and 2 drops per affected ear on the second day. The child remained free of auricular infection (otitis media and/or otitis externa) for several years.

### Example 9

A solution of polyvinylpyrolidone-iodine was prepared by adding 1 gram of Povadyne® powder (containing 10% total iodine) to 9 ml of a solution of 0.1% HA-0.1% polyvinylpyrolidone in buffered salt solution, to which had been added 0.1 ml of rose oil (called Rose Oil Natural, and in which 20% rose oil is dissolved in Tween 80). This preparation was a clear, brown solution with a mild, pleasant odor.

### Example 10

A solution of polyvinylpyrolidone-iodine was prepared by adding 1.5 grams of Povadyne® powder (containing 10% total iodine) to 8.5 ml of a solution of 0.1% HA-0.1% polyvinylpyrolidone in buffered salt solution, to which had been added 0.1 ml of rose oil (called Rose Oil Natural, and in which 20% rose oil is dissolved in Tween 80). This preparation had a total iodine concentration of 1.5% and was a clear, dark brown solution with a mild, pleasant odor.

### Example 11

This example illustrates a composition with 20% PVP-I in a buffered aqueous alcohol solution. To prepare 100 grams of this composition, 20 grams of PVP-I powder was dissolved in 40 grams of buffer solution composed of 0.08M sodium phosphate dibasic solution and mixed until the PVP-I crystals were dissolved. One gram of water soluble rose oil (called Rose Oil Natural, and in which 20% rose oil is dissolved in Tween 80) was added to the PVP-I-buffer solution. 4 grams of glycerin was dissolved in the PVP-I solution. The final pH of the composition is 3.78, and the solution is a dark brown, slightly viscous composition.

1. A topical pharmaceutical composition comprising a therapeutically effective amount of PVP-I having a concentration of iodine in the range of 0.5-5.0% by weight, a pharmaceutically acceptable excipient therefor and a pharmaceutically acceptable vehicle therefor.

2. A composition according to claim 1 wherein the pharmaceutically acceptable excipient is a natural oil or oil extract.

3. A composition according to claim 2 wherein the pharmaceutically acceptable oil or oil extract is oil or oil extract of clove, calendula, arnica, geranium, rose, wheat, chamomile or sea-buckhorn.

4. A composition according to claim 1 wherein the pharmaceutically acceptable vehicle is an oil selected from the group consisting of mineral oil, castor oil, sunseed oil,sesame oil, olive oil or a pharmaceutically acceptable polysaccharide.

5. A composition according to claim 4 wherein the polysaccharide is a glycosaminoglycan.

6. A composition according to claim 5 wherein the glycosaminoglycan is hyaluronic acid, chondroitin sulfate, heparin sulfate or dermatan sulfate.

7. A composition according to claim 5 wherein the glycosaminoglycan is hyaluronic acid.

8. A composition according to claim 7 wherein the hyaluronic acid is the chemically modified hyaluronic acid, hylan.

9. A composition according to Claim land further comprising an anti-inflammatory agent.

10. A composition according to claim 9 wherein the anti-inflammatory agent is cortisone or acetaminophen.

11. A composition according to claim 1 and further comprising an iodine odor masking agent.

12. A composition according to claim 11 wherein the odor-masking agent is eugenol, rose oil or clove oil.

13. A method of treating a mammal afflicted with otitis media comprising administering directly into the ear canal of such a mammal, a therapeutically effective amount of a composition as claimed in claim 1.

14. A method according to claim 13 wherein the therapeutically effective amount of the composition is 1-2 drops per day, per ear for not more than about 3 days.

15. A method of treating a mammal afflicted with sinusitis comprising intranasally administering a therapeutically effective dose of a composition as claimed in claim 1.

16. A method according to claim 15, wherein the therapeutically effective amount of the composition is 1-2 applications of nasal spray per day, per nostril.
